(19) United States
(12) Patent Application Publication
(10) Pub. No.: US 2021/0022585 A1
(43) Pub. Date: Jan. 28, 2021

(54) ENDOSCOPE SYSTEM, ENDOSCOPE CONTROL DEVICE, OPERATING METHOD OF ENDOSCOPE SYSTEM, AND NON-TRANSITORY COMPUTER-READABLE RECORDING MEDIUM STORING ENDOSCOPE CONTROL PROGRAM

(71) Applicant: OLYMPUS CORPORATION, Tokyo (JP)

(72) Inventor: Hirokazu NISHIMURA, Tokyo (JP)

(73) Assignee: OLYMPUS CORPORATION, Tokyo (JP)

(21) Appl. No.: 16/986,961

(22) Filed: Aug. 6, 2020

Related U.S. Application Data
(63) Continuation of application No. PCT/JP2018/004653, filed on Feb. 9, 2018.

(51) Int. Cl. A61B 1/00; G06T 7/00; A61M 25/01

(52) U.S. Cl. CPC A61B 1/0016; G06T 7/0012; A61M 25/0155; A61M 25/0113; G06T 2207/10068; A61M 2025/0166; A61B 1/00006

(57) ABSTRACT

An endoscope system includes: a insertion section to be inserted into a target; an imaging element to image the target; a drive section to conduct an insertion; a determination section to determine an insertion condition based on image information and insertion state information; a generation section to generate insertion control information based on a determination result; and a control section to control the drive section based on the insertion control information. The determination section determines whether an insertion can be continued by comparing newly acquired image information or newly acquired insertion state information with corresponding information stored in the storage. The generation section generates halt information when it is determined that the insertion cannot be continued.

### CROSS-REFERENCE TO RELATED APPLICATIONS

This application is a Continuation Application of PCT Application No. PCT/JP2018/004653, filed Feb. 9, 2018, the entire contents of which are incorporated herein by reference.

### BACKGROUND OF THE INVENTION

1. Field of the Invention

The present invention relates to an endoscope system, an endoscope control device, an operating method of endoscope system, and a non-transitory computer-readable recording medium storing endoscope control program.

2. Description of the Related Art

An endoscope automated insertion system, which can automatically operate the insertion of an endoscope into the examination target with a control device instead of a manipulator's manual operation, has been known. For instance, Japanese Patent No. 4323515 discloses an endoscope system including an insertion section having a bendable section; a bend drive section for changing the direction of the bendable section and a forward/backward drive section for moving the insertion section forward or backward; a control device for controlling the bend drive section and forward/backward drive section. This system further includes an endoscope shape detection device for detecting a bent shape of the insertion section. In this system, the control device moves the insertion section forward or backward, while correcting the movement direction of the insertion section based on the bent shape detected by the endoscope shape detection device.

### BRIEF DESCRIPTION OF THE DRAWINGS

An endoscope system includes: a flexible insertion section to be inserted into an examination target; an imaging element configured to image the examination target; a drive section configured to conduct an insertion operation of the insertion section; an image processing circuit configured to generate image information based on an examination target image obtained by the imaging element; a condition determination section configured to acquire the image information and insertion state information relating to an insertion state of the insertion section and determine an insertion condition based on the acquired image information and insertion state information; a storage configured to store the acquired image information and insertion state information; an insertion control information generation section configured to generate insertion control information based on a determination result obtained by the condition determination section; and a control section configured to control the drive section based on the insertion control information. The condition determination section determines whether or not an insertion of the insertion section can be continued by comparing at least one of newly acquired image information and newly acquired insertion state information with corresponding information stored in the storage. When the condition determination section determines that the insertion cannot be continued, the insertion control information generation section generates halt information.

An endoscope control device includes: a condition determination section configured to acquire image information and insertion state information relating to an insertion state of an insertion section of an endoscope, and determine an insertion condition based on the acquired information; a storage configured to store the acquired information; an insertion control information generation section configured to generate insertion control information for controlling an insertion operation of the insertion section, based on a determination result obtained by the condition determination section; and a control section configured to control the insertion operation of the insertion section based on the insertion control information. The condition determination section determines whether or not an insertion of the insertion section can be continued by comparing at least one of newly acquired image information and newly acquired insertion state information with the information stored in the storage. When the condition determination section determines that the insertion cannot be continued, the insertion control information generation section generates halt information.

An endoscope system includes a flexible insertion section to be inserted into an examination target, an imaging element configured to image the examination target, and a drive section configured to conduct an insertion operation of the insertion section. A method of operating the endoscope system includes: generating image information at an image processing circuit, based on information obtained by the imaging element; acquiring the image information and insertion state information relating to an insertion state of the insertion section, and determining an insertion condition based on the acquired information, at a condition determination section; storing the acquired information in a storage; generating insertion control information at an insertion control information generation section, based on a determination result obtained by the condition determination section; and controlling, at a control section, the drive section based on the insertion control information. The method includes determining at the condition determination section whether or not an insertion of the insertion section can be continued, by comparing at least one of newly acquired image information and newly acquired insertion state information with the information stored in the storage. The method includes generating halt information at the insertion control information generation section when it is determined at the condition determination section that the insertion cannot be continued.

A non-transitory computer-readable storage medium stores a program to cause a computer to function as: a condition determination section that acquires image information and insertion state information relating to an insertion state of an insertion section of an endoscope, and determines an insertion condition based on the acquired information; a storage that stores the acquired information; an insertion control information generation section that generates insertion control information to control an insertion operation of the insertion section based on a determination result obtained by the condition determination section; and a control section that controls the insertion operation of the insertion section based on the insertion control information. The program causes the condition determination section to determine whether or not an insertion of the insertion section can be continued by causing the condition determination section to compare at least one of newly acquired image information and newly acquired insertion state information with the information stored in the storage. When it is determined by the condition determination section that the insertion cannot be continued, the program causes the insertion control information generation section to generate halt information.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

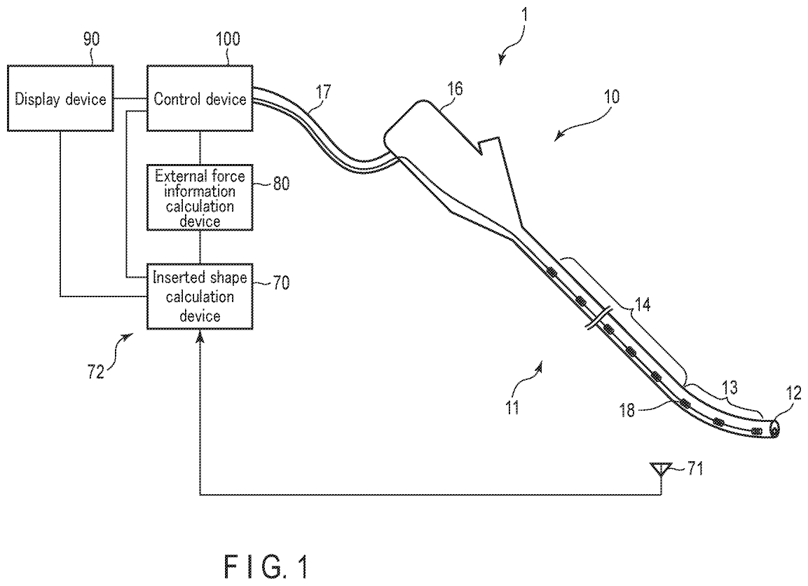

FIG. 1 is a diagram schematically showing an exemplary endoscope system.

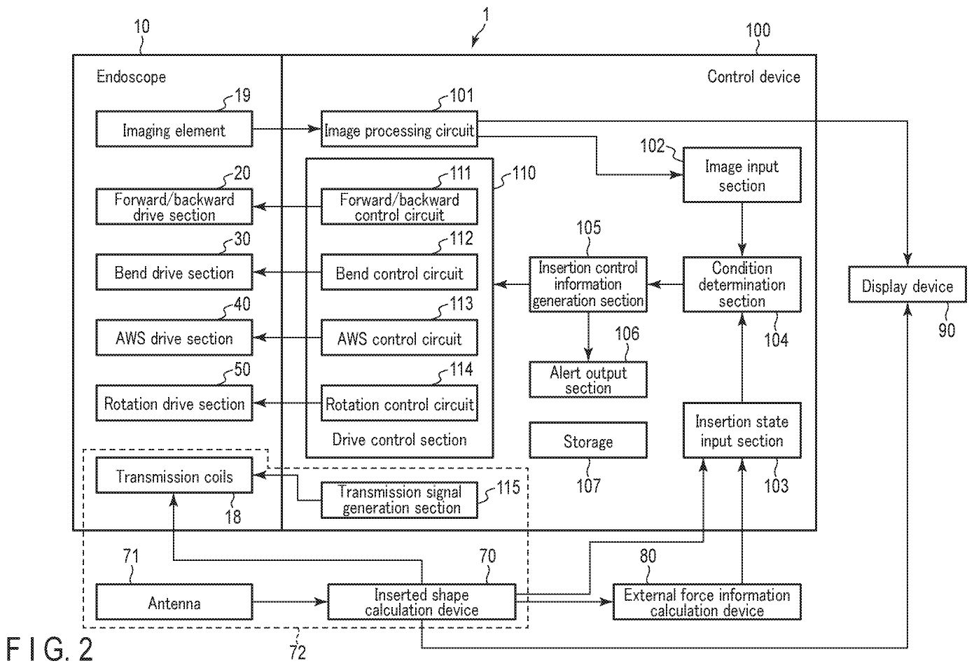

FIG. 2 is a block diagram showing an exemplary structure of the endoscope system.

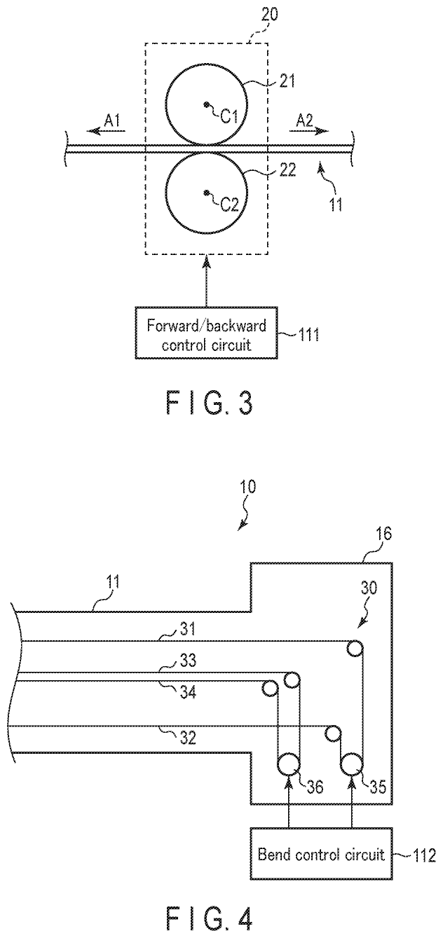

FIG. 3 is a diagram showing an exemplary structure of a forward/backward drive section for automated insertion of the endoscope system.

FIG. 4 is a diagram showing an exemplary structure of a bend drive section for automated insertion of the endoscope system.

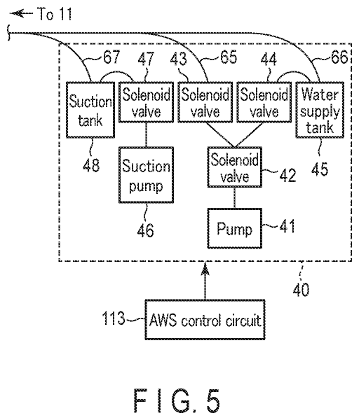

FIG. 5 is a diagram showing an exemplary structure of an AWS drive section for automated insertion of the endoscope system.

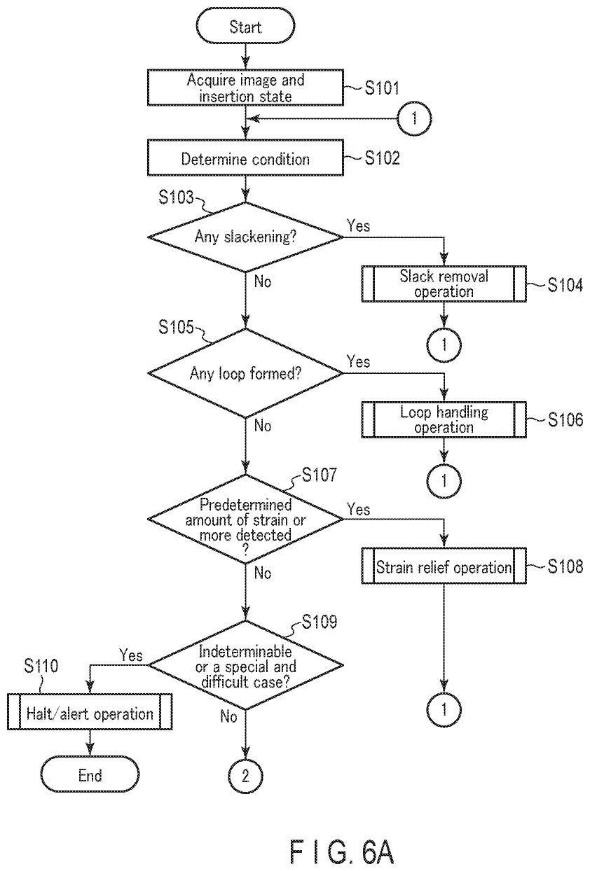

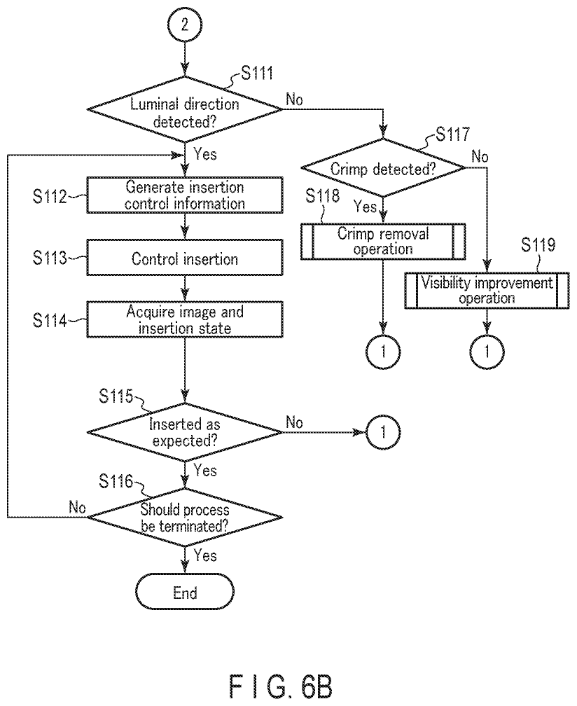

FIG. 6A is a diagram showing an exemplary operation of the endoscope system at the time of insertion.

FIG. 6B is a diagram showing an exemplary operation of the endoscope system at the time of insertion.

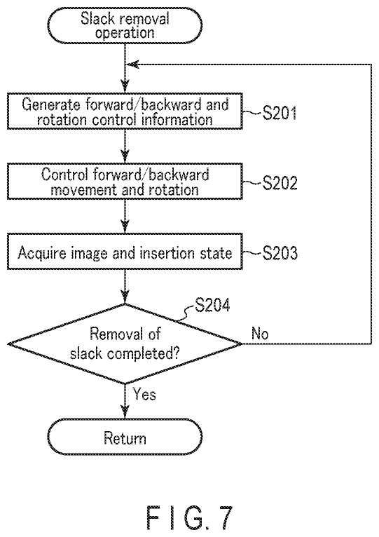

FIG. 7 is a diagram showing an exemplary slack removal operation.

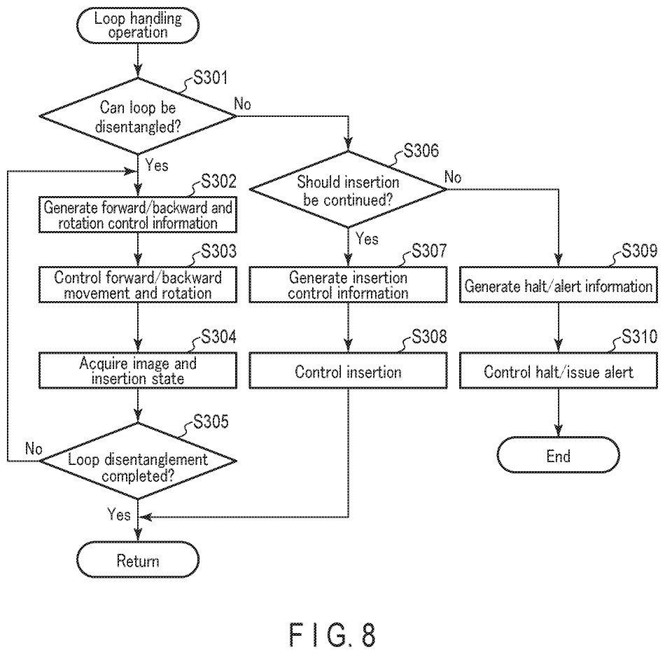

FIG. 8 is a diagram showing an exemplary loop handling operation.

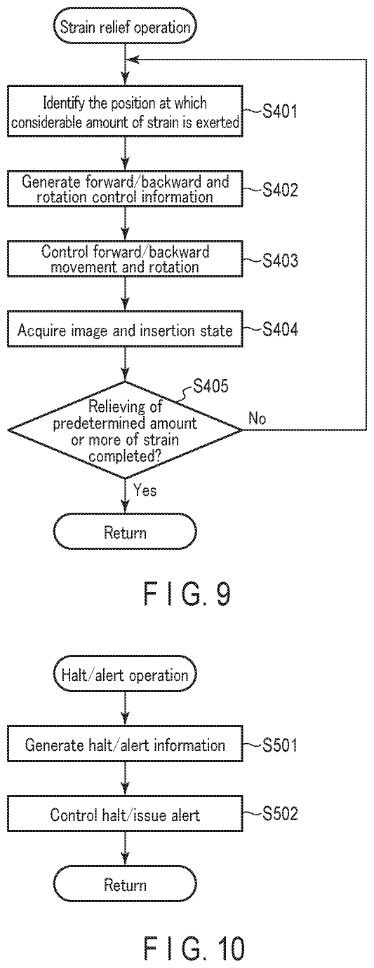

FIG. 9 is a diagram showing an exemplary strain relief operation.

FIG. 10 is a diagram showing an exemplary halt/alert operation.

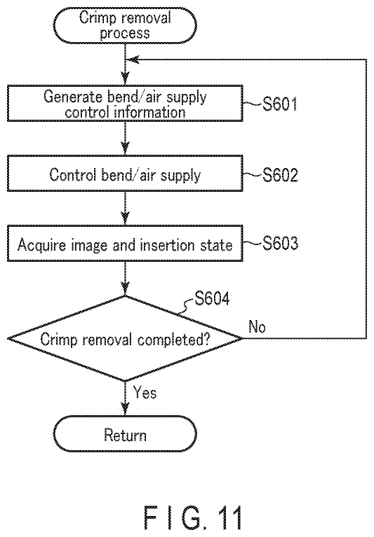

FIG. 11 is a diagram showing an exemplary crimp removal operation.

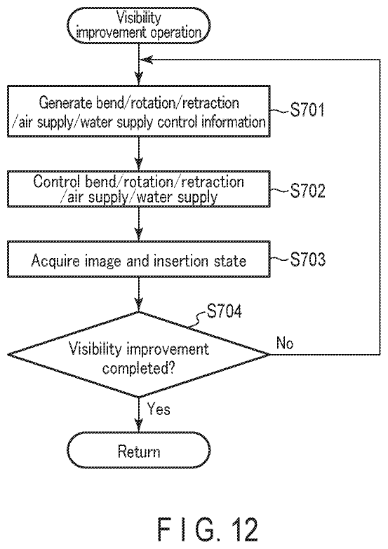

FIG. 12 is a diagram showing an exemplary visibility improvement operation.

### DETAILED DESCRIPTION OF THE INVENTION

Embodiments of the present invention will be described below, with reference to the drawings.

FIG. 1 is a diagram schematically showing an example of an endoscope system 1 according to an embodiment of the present invention. FIG. 2 is a block diagram showing an exemplary structure of the endoscope system 1. FIG. 3 is a diagram showing an exemplary structure for automated insertion of the endoscope system 1. The endoscope system 1 includes an endoscope 10, an inserted shape calculation device 70, an external force information calculation device 80, a display device 90, and a control device 100. The inserted shape calculation device 70 and external force information calculation device 80 constitute an insertion state detection section.

### Endoscope

The endoscope 10 includes an insertion section 11 and a control section 16. The insertion section 11 has an elongated tubular body having a distal end and a proximal end, and is to be inserted into an examination target. The insertion section 11 includes a distal end section 12, a bendable section 13, and a flexible tube section 14 in this order from the distal end. The distal end section 12 includes an illumination optical system and observation optical system, which are not shown in the drawings, and an imaging element 19 illustrated in FIG. 2. The bendable section 13 is bent by a bend drive section 30, which will be described later, in a desired direction (e.g., upward/downward leftward/rightward). The flexible tube section 14 is a soft tube that can freely bend. The control section 16 is positioned on the proximal end side of the insertion section 11 in the endoscope 10. The control section 16 includes part of the drive sections 20, 30, 40, and 50, which will be described later.

An image transmission cable and light guide, which are not shown in the drawings, are provided inside the insertion section 11. A universal cord 17 extends from the control section 16, and includes the image transmission cable and light guide, as well as electric cables from various drive sections 20, 30, 40, and 50. The endoscope 10 is connected to the control device 100 by way of the universal cord 17.

Transmission coils 18 are arranged inside the insertion section 11, for example in the bendable section 13 and flexible tube section 14. The transmission coils 18 are arranged apart from each other in the longitudinal direction (axial direction) of the insertion section 11.

Under the control of the control device 100, the endoscope 10 moves the insertion section 11 forward and backward in the examination target (forward/backward operation). Furthermore, under the control of the control device 100, the bendable section 13 can be bent in a desired direction (bend operation). Under the control of the control device 100, air and water can be supplied to the air supply inlet and water supply inlet provided in the distal end section 12, which are not illustrated in the drawings, and can be sucked through the suction opening (air supply (A), water supply (W), and suction (S) operation; hereinafter referred to as “AWS operation”). Moreover, under the control of the control device 100, the insertion section 11 can be rotated around the insertion axis (rotation operation).

The endoscope 10 according to the present embodiment is a fully automated insertion endoscope. The endoscope 10, however, may be a semi-automated insertion endoscope or a partially automated insertion endoscope. Here, semi-automation, partial automation, and full automation may be defined as follows.

Semi-automation: For example, the bend operation may be automated based on the determination of the control device 100 instead of the manipulator's determination and input, while the forward/backward operation may be conducted based on the manipulator's determination and input. The control device 100 controls all but at least one of the insertion operations including the forward/backward operation, bend operation, AWS operation, and rotation operation.

Partial automation: In normal situations (where the insertion operation can be smoothly conducted, for example), the insertion operation is conducted based on the manipulator's determination and input. On the other hand, in specific situations that make the insertion operation difficult to continue (where a loop is created in the insertion section 11, for example), the control device 100 controls the insertion operation.

Full automation: All of the basic insertion operations including the forward/backward operation, bend operation, AWS operation, and rotation operation are conducted by the control device 100.

In the case of semi-automation or partial automation, the manipulator inputs an operation instruction by way of an input device equipped in the control section 16 such as a manipulation knob, manipulation buttons, joystick, and foot switch, which are not shown in the drawings, to the control device 100.

The endoscope 10 includes a forward/backward drive section 20, a bend drive section 30, an AWS drive section 40, and a rotation drive section 50. The forward/backward drive section 20 includes a motor or the like, which is not shown in the drawings, and serves as a drive mechanism to move the insertion section 11 forward and backward. The forward/backward drive section 20 may be a mechanism for moving the insertion section 11 forward into the examination target by pushing the insertion section 11, and removing the insertion section 11 from the examination target by pulling the insertion section 11. In other words, the forward/backward drive section 20 is a mechanism for pushing and pulling the insertion section 11. The bend drive section 30 includes a motor, which is not illustrated in the drawings, and is a drive mechanism for bending the bendable section 13. The AWS drive section 40 is a drive mechanism for causing the endoscope 10 to supply air and water and to perform a suction. The rotation drive section 50 is a drive mechanism for twisting the insertion section 11 around the insertion axis to the left or right, or in other words for rotating the insertion section 11. That is, the endoscope 10 is an electronic endoscope that performs respective insertion operations with the drive sections 20, 30, 40, and 50.

The control device 100 includes a drive control section 110. The drive control section 110 includes a forward/backward control circuit 111, a bend control circuit 112, an AWS control circuit 113, and a rotation control circuit 114. The forward/backward drive section 20 is controlled by the forward/backward control circuit 111. The bend drive section 30 is controlled by the bend control circuit 112. The AWS drive section 40 is controlled by the AWS control circuit 113. The rotation drive section 50 is controlled by the rotation control circuit 114.

Exemplary structures of the drive sections 20, 30, and 40 will be explained below with reference to FIGS. 3 to 5.

As illustrated in FIG. 3, the forward/backward drive section 20 may include a pair of rollers 21 and 22. The rollers 21 and 22 are arranged to face each other across the insertion section 11. The rollers 21 and 22 are rotatable around rotational axes Cl and C2, respectively, by a motor, which is not shown in the drawings, where the motor is electrically connected to the forward/backward control circuit 111. Through the rotation of the rollers 21 and 22, the insertion section 11 can be sent in the direction indicated by arrow A1 toward the distal end, and pulled back in the direction indicated by arrow A2 toward the proximal end.

As illustrated in FIG. 4, the bend drive section 30 may include wires 31, 32, 33, and 34. The distal ends of these wires 31, 32, 33, and 34 are fixed to the top/bottom and left/right, respectively, of the bendable section 13. The proximal ends of the wires 31 and 32, which are not shown in the drawings, are coupled to the first pulley 35, while the proximal ends of the wires 33 and 34, which are not shown in the drawings, are coupled to the second pulley 36. The pulleys 35 and 36 are rotatable around their rotational shaft by the not-shown motor electrically connected to the bend control circuit 112. Through this rotation, the bendable section 13 can be bent upward, downward, leftward and rightward.

As illustrated in FIG. 5, the AWS drive section 40 includes a pump 41, solenoid valves 42, 43, and 44, a water supply tank 45, a suction pump 46, a solenoid valve 47, and a suction tank 48. The pump 41, solenoid valves 42, 43, and 44, suction pump 46, and solenoid valve 47 are electrically connected to the AWS control circuit 113.

An air supply duct 65 and a water supply duct 66 are arranged inside the insertion section 11 to extend in the longitudinal direction of the insertion section 11. The air supply duct 65 and water supply duct 66 are connected respectively to the air supply inlet and water supply inlet arranged in the distal end section 12, which are not shown in the drawings. The air supply duct 65 is connected to the pump 41 by way of the solenoid valve 43 and solenoid valve 42. The water supply duct 66 is provided in the water within the water supply tank 45 and connected to the pump 41 by way of the solenoid valve 44 and the solenoid valve 42. The AWS control circuit 113 supplies air and water by controlling the operations of the pump 41, solenoid valves 42, 43, and 44. When the solenoid valve 42 is closed, the air from the pump 41 leaks to the outside. When the solenoid valve 42 is opened toward the solenoid valve 43 only, with the solenoid valve 43 opened and the solenoid valve 44 closed, the air supplied from the pump 41 is sent through the air supply inlet to the outside by way of the air supply duct 65. With the air supplied, the examination target can be expanded. When the solenoid valve 42 is opened toward the solenoid valve 44 only, with the solenoid valve 43 closed and the solenoid valve 44 opened, the air supplied from the pump 41 pressurizes the water supply tank 45 so that the water inside the water supply tank 45 is sent through the water supply inlet to the outside by way of the water supply duct 66. With the water supplied, bubbles and residues in the distal end section 12 and soil on the lens and the like can be removed.

A suction duct 67 is provided inside the insertion section 11 to extend in the longitudinal direction of the insertion section 11. The suction duct is connected to the suction opening, which is not shown in the drawings, in the distal end section 12. The suction duct 67 is connected to the suction pump 46 by way of the suction tank 48 and solenoid valve 47. The AWS control circuit 113 conducts suction by controlling the operations of the suction pump 46 and solenoid valve 47. When the solenoid valve 47 is closed, the suction pump 46 sucks ambient air, while when the solenoid valve 47 is opened, suction is conducted through the suction opening by way of the suction tank 48 and suction duct 67. With the suction, liquids such as residues can be removed from the distal end section 12.

The above described structures of the forward/backward drive section 20, bend drive section 30, and AWS drive section 40 are described as mere examples, but should not be limited thereto. Various forms of the drive mechanism may be adopted. For instance, the forward/backward drive section 20 may be a propulsion mechanism using inflation/contraction of a balloon, or a propulsion mechanism incorporating a propulsion generator rotatably arranged on the outer periphery of the insertion section in the longitudinal direction as disclosed in Japanese Patent No. 4864003. Furthermore, the forward/backward drive section 20 may be a mechanism that includes a grip portion, which a person can grip with a hand to hold the insertion section 11, and that can be gripped and un-gripped to push and pull the insertion section 11 gripped at the grip portion.

The rotation drive section 50 may include a gripping portion for gripping the insertion section 11 on the proximal side of the insertion section 11 and a motor for supplying a driving force to the gripping portion, although these are not shown in the drawings. In the rotation drive section 50, the gripping portion turns the insertion section 11 to the left or to the right around the insertion axis with the driving force supplied from the motor. That is, the rotation drive section 50 twists the insertion section 11 to the left or right. The rotation drive section 50 is not limited thereto, and various types of drive mechanisms that can turn the insertion section 11 can be adopted. The rotation drive section 50 is electrically connected to the rotation control circuit 114. The rotation control circuit 114 controls, for example, the rotation angle, rotation speed, and rotation force (torque) of the insertion section 11.

### Inserted Shape Detection Device

According to the present embodiment, the inserted shape calculation device 70 functions as an inserted shape detection device 72 configured to detect as shape information at least part of the shape of the insertion section 11, together with the transmission coils 18, the antenna 71, and a transmission signal generation section 115 of the control device 100.

The transmission signal generation section 115 generates signals to generate a magnetic field from the transmission coils 18, such as sinusoidal currents. The generated signals are output from the transmission coils 18 on the distal end side of the insertion section 11, in the order determined for individual transmission coils 18. Each of the transmission coils 18 generates a magnetic field with a current flowing from the transmission signal generation section 115.

The antenna 71 is constituted by reception coils, which are not shown in the drawings. The antenna 71 detects the magnetic field signals generated by the transmission coils 18. The inserted shape calculation device 70 and the antenna 71 are connected in a wired or wireless manner.

The inserted shape calculation device 70 calculates positional information that includes the positions and directional vectors of respective transmission coils 18, based on the magnetic field intensity information input from the antenna 71. The inserted shape calculation device 70 calculates the shape information of the insertion section 11 based on the positional information of the individual transmission coils 18. Furthermore, the inserted shape calculation device 70 is also capable of calculating the length of the insertion of the insertion section 11 into the examination target, or in other words the insertion length information of the insertion section 11 with respect to the examination target. The shape information and insertion length information are output to the external force information calculation device 80 and control device 100. The shape information and insertion length information may be output to the display device 90 in a displayable format.

The inserted shape detection device may be of any type other than a magnetic type, as long as the device can detect the bent state of the insertion section 11 in order to detect the shape of at least part of the insertion section 11. For instance, one of sensing using magnetism (magnetic sensor), sensing using ultrasound (ultrasonic sensor), sensing using optical loss of the light guided through optical fibers (optical fiber sensor), sensing using distortion (distortion sensor), or sensing using an x-ray absorption material, or any combination thereof, can be adopted.

### External Force Information Calculation Device

The external force information calculation device 80 calculates, based on the shape information of the insertion section 11 detected by the inserted shape calculation device 70, the information of an external force applied to the insertion section 11 at different positions thereof along the longitudinal direction. The external force information calculation device 80 may store in advance the data of curvatures (or curvature radii) and bent angles at predetermined positions of the insertion section 11 without external force being applied, and the data of curvatures (or curvature radii) and bent angles at the predetermined positions of the insertion section 11 obtained by applying a predetermined amount of external force to the predetermined positions of the insertion section 11 from every possible direction. By referring to the prestored data of various types, the external force information calculation device 80 may calculate external force information relating to the strength and direction of the external force at the position of each transmission coil 18, based on the curvature (or curvature radius) and bent angle of the insertion section 11 at the position of the transmission coil 18. For the external force information calculation device 80, a scheme of calculation of the external force information as disclosed in Japanese Patent No. 5851204 or 5897092 may be adopted.

The external force information of the external force applied to the positions of the insertion section 11 in the longitudinal direction may be calculated by providing the insertion section 11 with a distortion sensor, pressure sensor, acceleration sensor, gyro sensor, wireless element, and the like.

### Control Device

The control device 100 includes an image processing circuit 101, an image input section 102, an insertion state input section 103, a condition determination section 104, an insertion control information generation section 105, an alert output section 106, and a drive control section 110. As mentioned earlier, the drive control section 110 includes a forward/backward control circuit 111, a bend control circuit 112, an AWS control circuit 113, and a rotation control circuit 114. These components are constituted by a processor such as a CPU that includes one or more integrated circuits. Alternatively, a software program for causing a computer processor to function as a control device 100 may be prepared in a storage 107, which will be described later, or in another storage medium so that the function of this control device 100 can be implemented by the processor when the processor executes this program.

The above described components of the control device 100 may be included in a control device different from the control device 100. For instance, the image input section 102, insertion state input section 103, condition determination section 104, insertion control information generation section 105, and drive control section 110 may be included in a control device different from an endoscopic video image processor that includes the image processing circuit 101. Alternatively, the components may be included in separate control devices. That is, a processor or hardware circuit that functions as each of the components of the control device 100 may be provided in a single housing or in multiple housings, as long as the functions of these components can be implemented.

The image processing circuit 101 converts an electric signal that has been converted from the light received from the examination target by the imaging element 19 of the distal end section 12 of the endoscope 10, to a video signal, creates an endoscopic image based on the examination target image, and displays an endoscopic image on the display device 90.

The endoscopic image information (hereinafter simply referred to as “image information”) generated by the image processing circuit 101 is input into the image input section 102. The inserted shape information and insertion length information calculated by the inserted shape calculation device 70, and the external force information calculated by the external force information calculation device 80 are input into the insertion state input section 103.

The condition determination section 104 acquires image information from the image input section 102 and insertion state information from the insertion state input section 103. The condition determination section 104 determines the insertion condition of the endoscope 10 based on at least one of the acquired image information and insertion state information.

Based on the insertion condition determined by the condition determination section 104, the insertion control information generation section 105 generates the insertion control information (forward/backward control information, bend control information, AWS control information, or rotation control information) as information for controlling the insertion operation. The alert output section 106 outputs an alert based on the insertion condition determined by the condition determination section 104.

In the drive control section 110, the forward/backward control circuit 111 operates the forward/backward drive section 20 based on the forward/backward control information generated by the insertion control information generation section 105. The bend control circuit 112 operates the bend drive section 30 based on the bend control information generated by the insertion control information generation section 105. The AWS control circuit 113 operates the AWS drive section 40 based on the AWS control information generated by the insertion control information generation section 105. The rotation control circuit 114 operates the rotation drive section 50 based on the rotation control information generated by the insertion control information generation section 105.

The control device 100 includes the storage 107. The storage 107 may be a semiconductor memory, for example. The storage 107 stores various types of data used by the insertion control information generation section 105 to generate the insertion control information, and various programs necessary for the operations of the control device 100.

### Display Device

The display device 90 is a monitor such as a liquid crystal display. The display device 90 displays endoscopic images, the shape of the inserted endoscope, and the like.

### Operation of Endoscope Automatic Insertion System

The operation of the control device 100 during a colonoscopy will be explained below by referring to FIGS. 6A and 6B. In the explanation below, the examination target is a large intestine, and the endoscope 10 is a large intestine endoscope.

At step S101, image information is input to the image input section 102 of the control device 100. Furthermore, the insertion state information of the insertion section 11 of the endoscope 10 is input to the insertion state input section 103 of the control device 100. The insertion state information to be input includes the inserted shape information and insertion length information from the inserted shape calculation device 70 and external force information from the external force information calculation device 80. The condition determination section 104 acquires the image information and insertion state information from the image input section 102 and insertion state input section 103, respectively.

At step S102, the condition determination section 104 determines the insertion condition, based on at least one of the image information and insertion state information acquired at step S101. In this exemplary case, the insertion condition is determined based on both types of information. According to the present embodiment, the insertion conditions observed at the time of the insertion operation for colonoscopy may be categorized into:

(i) insertion achieved with no serious problem raised
(ii) the insertion section 11 slackening
(iii) a loop formed in the insertion section 11
(iv) low visibility of the moving direction in the lumen or the like due to the intestinal tract being crimped
(v) luminal direction being lost
(vi) a considerable amount of strain being exerted on the insertion section 11
(vii) other cases (indeterminable, or particular and difficult cases etc.)

In the case of (i), the insertion section 11 can be moved forward as-is. In the cases of (ii) through (vii), certain events have occurred to obstruct the movement of the insertion section 11. The condition determination section 104 determines that the insertion section 11 is in the state of being able to move forward or in the state of some event having arisen that obstructs the movement of the insertion section 11.

The conditions relating to the shape of the insertion section 11 ((ii) and (iii)) are determined based on the inserted shape information. For instance, for the detection of a presence/absence of slack in the insertion section 11, a scheme utilizing the calculation of a slack amount from the analysis of the shape of the insertion section 11 as disclosed in Japanese Patent No. 4656988 may be adopted. For the detection of a loop and determination of a loop type, various schemes may be adopted. A scheme as disclosed in Japanese Patent No. 4274854 may be adopted, in which specific positions of the insertion section 11 are detected and stored in the storage 107 in a time series so that the analysis of the loop shape and determination of a loop type can be conducted upon the insertion section 11 based on the stored time-series positional information. An identification process adopting a deep neural network or the like by using training data in which a loop shape is categorized as an identification target, with the inserted shape information provided as input data, is also applicable.

The conditions relating to the moving direction and luminal direction ((iv) and (v)) are determined based on the image information. For instance, the endoscope 10 may take endoscopic images at the rate of 30 fps so that the condition determination section 104 can determine the condition using the image information corresponding to 10 seconds prior to the current time point, which means 300 frames. The endoscopic images to be input are not limited thereto, and the number of frames may be reduced from 30 frames per second by way of sampling. Various schemes may be adopted for detection of the luminal direction. For the detection of the luminal direction, various schemes can be adopted. In addition to the detection scheme based on the evaluation of changes in contrast in the endoscopic image, an image region dividing scheme adopting deep learning such as a fully convolutional network (FCN) may also be adopted.

The determination of a considerable amount of strain (an amount of strain larger than or equal to a predetermined amount) exerted on the insertion section 11 ((vi)) may be based on the external force information. A considerable amount of strain exerted indicates that the intestinal tract is under a load.

Insertion achieved without a serious problem ((i)) and other cases (indeterminable, or special and difficult cases; (vii)) are determined based on overall evaluation of the insertion state information and image information. In special and difficult cases, even an experienced surgeon may have difficulty in inserting the endoscope into the intestine for some reason. This may include cases of patients having narrowing or adhesion due to inflammatory bowel disease or laparotomy, and patients with diverticulosis. The cases of (vii) may include failure to obtain definite identification results from various identification schemes for the condition, and insertion operation being unable to continue due to discovery of narrowing or adhesion based on the image information. The cases of (vii) may also include no improvement observed in the condition even after the detection and measurement operations of the condition, the recurrence of the same condition such as any of the conditions (ii) to (vi) recurring after the condition is temporarily resolved, and the distal end of the insertion section 11 being unable to move forward for some reason. Such conditions can be determined by storing the insertion state information and image information in the storage 107 each time the information is acquired and comparing in the condition determination section 104 the stored information with newly acquired insertion state information and image information. Alternatively, the determination can be made by arranging a counter to count the number of times (occurrence frequency) that the same condition occurs or that the same condition recurs within a predetermined length of time.

In this manner, at step S102, the condition determination section 104 determines which of the conditions (i) to (vii) the insertion section 11 corresponds to. Thereafter, in the following steps, the condition determination section 104 makes a determination based on the insertion condition determined at step S102.

At step S103, the condition determination section 104 determines whether or not slackening is occurring in the insertion section 11 (i.e., whether or not the determination result is (ii)) based on the determination result obtained at step S102. If it is determined that there is slack (“yes” at step S103), the process proceeds to step S104, where the slack removal operation is executed. If it is determined that there is no slack (“no” at step S103), the process proceeds to step S105.

FIG. 7 is a flowchart showing an exemplary slack removal operation. In the slack removal operation, at step S201, the insertion control information generation section 105 generates forward/backward and rotation control information, which is the information for the forward/backward and rotation control that is to be performed by the drive control section 110 to remove the slack of the insertion section 11.

To remove slack, a pulling operation works effectively. Repeating the pushing and pulling operations for approximately 2 to 3 centimeters may also work effectively. Such an operation may be referred to jiggling. If the slack cannot be removed even by this operation, a rotating operation (twisting operation) approximately 45° to the left and to the right may be effective. The insertion control information generation section 105 acquires operation-related information stored in the storage 107, or operation-related information from other external storage devices, and generates the forward/backward and rotation control information.

At step S202, the forward/backward control circuit 111 or rotation control circuit 114 of the drive control section 110 operates the forward/backward drive section 20 or rotation drive section 50, based on the forward/backward and rotation control information generated at step S201. In this manner, the forward/backward and rotation control is implemented upon the insertion section 11.

At step S203, the image information is input to the image input section 102. Furthermore, the insertion state information of the insertion section 11 of the endoscope 10 is input to the insertion state input section 103. The condition determination section 104 acquires the image information and insertion state information from the image input section 102 and insertion state input section 103.

At step S204, the condition determination section 104 determines whether the removal of slack from the insertion section 11 is completed. The operations of steps S201 through S204 are repeated until the completion of the slack removal is determined. When it is determined that the slack removal is completed (“yes” at step S204), the slack removal operation returns.

After the slack removal operation in step S104, the process returns to step S102.

At step S105, the condition determination section 104 determines whether or not a loop is formed in the insertion section 11 (i.e., whether the determination result is (iii)) based on the determination result obtained at step S102. When it is determined that a loop is formed (“yes” at step S105), the process proceeds to step S106, where a loop handling operation is executed. When it is determined that no loop is formed (“no” at step S105), the process proceeds to step S107.

FIG. 8 is a flowchart showing an exemplary loop handling operation. In the loop handling operation, at step S301, the condition determination section 104 determines whether the loop can be disentangled. For instance, if the distal end section 12 is inserted sufficiently deep (for a predetermined length or greater) into the large intestine (in most cases, up to the descending colon or the splenic flexure) with respect to the loop, it is determined that the loop can be disentangled.

When it is determined that the loop can be disentangled (“yes” at step S301), the process proceeds to step S302. At step S302, the insertion control information generation section 105 generates forward/backward and rotation control information, which is information for the forward/backward and rotation control performed by the drive control section 110 to disentangle the loop in the insertion section 11.

Loops that may be formed in the insertion section 11 in the vicinity of the sigmoid colon during colonoscopy include an α loop, a reverse-a loop, an N loop, and a γ loop. The insertion control information generation section 105 generates forward/backward and rotation control information for the removal of respective loops. The insertion control information generation section 105 may read information relating to the operation for removing various types of loops from the storage 107 or any other storage device, and generate the forward/backward and rotation control information. For suitable operations for the removal of various types of loops, for example, the technique disclosed in “(Sonyu o Yoi ni Surutameno Kufu) Naishikyo Sonyu Keijo Kansoku Sochi no Katsuyo (Utilization of Endoscope Insertion Shape Observation Apparatus (for facilitation of insertion))” Takayoshi Suzuki and 10 others, Endoscopia Digestiva, Tokyo Igakusha, published on Apr. 25, 2016, Vol. 28, No. 4, pp. 592-596 may be referred to.

At step S303, the forward/backward control circuit 111 or rotation control circuit 114 of the drive control section 110 operates the forward/backward drive section 20 or rotation drive section 50, based on the forward/backward and rotation control information generated at step S302. In this manner, the forward/backward and rotation control is implemented upon the insertion section 11.

At step S304, image information is input into the image input section 102. Furthermore, the insertion state information of the insertion section 11 of the endoscope 10 is input into the insertion state input section 103. The condition determination section 104 acquires the image information and insertion state information from the image input section 102 and insertion state input section 103.

At step S305, the condition determination section 104 determines whether the loop disentanglement of the insertion section 11 is completed. The operations of steps S302 through S305 are repeated until it is determined that the loop entanglement is completed. When it is determined that the loop entanglement is completed (“yes” at step S305), the loop handling operation returns.

On the other hand, when it is determined at step S301 that the loop cannot be disentangled (“no” at step S301), the process proceeds to step S306. At step S306, the condition determination section 104 determines whether or not the insertion should be continued. For instance, if the condition is such that the insertion should be continued without disentangling the loop (e.g., if the distal end of the insertion section 11 reaches the splenic flexure), the condition determination section 104 determines that the insertion should be continued as-is even if the loop cannot be disentangled. When it is determined that the insertion should be continued (“yes” at step S306), the process proceeds to step S307. At step S307, the insertion control information generation section 105 generates the insertion control information, which is information for the insertion control to be performed by the drive control section 110.

At step S308, the drive control section 110 operates the forward/backward drive section 20, bend drive section 30, AWS drive section 40 or rotation drive section 50, based on the insertion control information generated at step S307. In this manner, the control of the insertion of the insertion section 11 is executed. After step S308, the loop handling operation returns.

On the other hand, when it is determined at step S306 that the insertion should not be continued (“no” at step S306), the process proceeds to step S309.

At step S309, the insertion control information generation section 105 generates halt/alert information. At step S310, the drive control section 110 halts the drive sections 20, 30, and 40. Furthermore, the alert output section 106 outputs an alert based on the alert information from the insertion control information generation section 105. After step S310, the process is terminated. After the halt/alert operation, a manual operation may be conducted by the surgeon to improve the situation.

After the loop handling operation returns from step S305 or S308, the process returns to step S102.

At step S107, the condition determination section 104 determines whether or not an amount of strain on the insertion section 11 that is larger than or equal to a predetermined amount is detected (i.e., whether the determination result is (vi)), based on the determination result obtained at step S102. When it is determined that an amount of strain larger than or equal to the predetermined amount is detected (“yes” at step S107), the process proceeds to step S108, where the strain relief operation is executed. When it is determined that an amount of strain larger than or equal to the predetermined amount is not detected (“no” at step S107), the process proceeds to step S109.

FIG. 9 is a flowchart showing an exemplary strain relief operation. In the strain relief operation, at step S401, the condition determination section 104 identifies the position of the insertion section 11 where a considerable amount of strain is exerted, based on the insertion state information, using a threshold processing or the like. At step S402, by referring to the identified position, the insertion control information generation section 105 generates the forward/backward and rotation control information, which is information for the control performed by the drive control section 110 to relieve the amount of strain. The forward/backward and rotation control information similar to that for the slack removal may be generated. At step S403, the forward/backward control circuit 111 or rotation control circuit 114 of the drive control section 110 operates the forward/backward drive section 20 or rotation drive section 50, based on the forward/backward and rotation control information generated at step S402. In this manner, the forward/backward and rotation control is implemented upon the insertion section 11. By implementing such an insertion operation, the resistance to the insertion section 11 can be relieved.

At step S404, the image information is input to the image input section 102. Furthermore, the insertion state information of the insertion section 11 of the endoscope 10 is input to the insertion state input section 103. The condition determination section 104 acquires the image information and insertion state information from the image input section 102 and insertion state input section 103.

At step S405, the condition determination section 104 determines whether or not the relieving of an amount of strain larger than or equal to a predetermined amount is completed. The operations of steps S401 through S405 are repeated until the relieving is completed. When it is determined that the relieving is completed (“yes” at step S405), the strain relief operation returns.

After the strain relief operation at step S108, the process returns to step S102.

At step S109, the condition determination section 104 determines whether or not the case is indeterminable or is a special and difficult case (i.e., whether the determination result is (vii)) based on the determination result obtained at step S102. When it is determined that the case is indeterminable or is a special and difficult case (“yes” at step S109), the process proceeds to step S110, where the halt/alert operation is conducted. When it is determined that the case is not indeterminable or is not a special and difficult case (“no” at step S109), the process proceeds to step S111.

FIG. 10 is a flowchart showing an exemplary halt/alert operation. At step S501, the insertion control information generation section 105 generates the halt/alert information. At step S502, the drive control section 110 halts the drive sections 20, 30, 40, and 50. Furthermore, the alert output section 106 outputs an alert based on the alert information from the insertion control information generation section 105. When the operation at step S502 is completed, the operation returns.

After the halt/alert operation at step S110, the process is terminated.

After the halt/alert operation, a manual operation may be conducted by the surgeon to improve the situation. For instance, after pulling the insertion section 11 approximately 10 centimeters toward the proximal side to retract it, the insertion section 11 may be reinserted. Alternatively, upon the judgment of the surgeon, operations such as the continuation or halt of the insertion, manual abdominal compression, or replacement with a finer insertion section in the endoscope may be suitably conducted.

At step S111, the condition determination section 104 determines whether or not the luminal direction is detected (i.e., whether the determination result is (i), or either one of (iv) and (v)). When the luminal direction is detected (“yes” at step S111), the process proceeds to step S112. This corresponds to the operation for (i), where the insertion has been achieved without any serious problems.

At step S112, the insertion control information generation section 105 generates insertion control information for the insertion control to be conducted. At step S113, the drive control section 110 operates the forward/backward drive section 20, bend drive section 30, AWS drive section 40, or rotation drive section 50, based on the insertion control information generated at step S112. In this manner, the control of the insertion of the insertion section 11 is executed.

At step S114, the image information is input into the image input section 102. Furthermore, the insertion state information of the insertion section 11 of the endoscope 10 is input to the insertion state input section 103. The condition determination section 104 acquires the image information and insertion state information from the image input section 102 and insertion state input section 103. At step S115, the condition determination section 104 determines whether the insertion section 11 is in the state of being inserted as expected (whether or not the insertion can be continued without any problem) through the insertion operation executed at step S113, based on one or more items of the information acquired at step S114. When it is determined that the insertion is as it should be (“yes” at step S115), the process proceeds to step S116. When it is determined that the insertion is not as it should be (“no” at step S115), the process returns to step S102.

At step S116, the condition determination section 104 determines whether or not the process should be terminated. This may be determined, for example, based on the presence/absence of a termination instruction (external input from an input device that is not shown in the drawings) based on the distal end of the insertion section 11 reaching the appendix. Alternatively, the determination may be made based on the insertion state information and image information. When it is determined that the process should not be terminated (“no” at step S116), the process returns to step S112. When it is determined that the process should be terminated (“yes” at step S116), the process is terminated.

On the other hand, at step S111, when it is determined that the luminal direction is not detected by the condition determination section 104 (“no” at step S111), the process proceeds to step S117. At step S117, the condition determination section 104 determines whether a crimp is detected (i.e., whether the determination result is (iv) or (v)). When a crimp is detected (“yes” at step S117), the process proceeds to step S118, where a crimp removal operation is executed. This corresponds to the operation for (iv), in which the movement direction in the lumen is not visible due to the crimped intestinal tract.

FIG. 11 is a flowchart showing an exemplary crimp removal operation. At step S601, the insertion control information generation section 105 generates information for bend/air supply control that is to be conducted to remove a crimp. If the luminal crimp due to gathered creases or the like is identified, it is effective to conduct a bend operation in a manner such that the insertion section 11 can face toward the crimp and then to supply air to expand the lumen. The insertion control information generation section 105 acquires operation-related information stored in the storage 107, or operation-related information from other external storage devices, and thereby generates the bend/air supply control information.

At step S602, the bend control circuit 112 and AWS control circuit 113 of the drive control section 110 operates the bend drive section 30 and AWS drive section 40 to control the bending and air supply, based on the generated bend/air supply control information.

At step S603, the image information is input to the image input section 102. Furthermore, the insertion state information of the insertion section 11 of the endoscope 10 is input to the insertion state input section 103. The condition determination section 104 acquires the image information and insertion state information from the image input section 102 and insertion state input section 103.

At step S604, the condition determination section 104 determines whether the removal of the crimp is completed.

The operations of steps S601 through S604 are repeated until the removal of the crimp is completed. When it is determined that the removal of the crimp is completed (“yes” at step S604), the crimp removal operation returns. After the crimp removal operation at step S118, the process returns to step S102.

On the other hand, if no crimp is detected at step S117 (“no” at step S117), the process proceeds to step S119, where a visibility improvement operation is executed. This corresponds to the operation for (v), in which the luminal direction is lost.

FIG. 12 is a flowchart showing an exemplary visibility improvement operation. At step S701, the insertion control information generation section 105 generates information for bend/rotation/retraction/air supply control to improve the visibility. For instance, when the luminal direction is not detected, searching by bend operations or rotation operations and improving visibility by retraction or air supply are effective. The insertion control information generation section 105 acquires operation-related information stored in the storage 107 or operation-related information from other external storage devices to generate the bend/rotation/retraction/air supply control information. In order to deal with soil on the surface of the distal end of the distal end section 12, water supply control information may be generated.

At step S702, the drive control section 110 operates the drive sections 20, 30, 40, and 50 to control the bend/rotation/retraction/air supply/water supply control, based on the generated bend/rotation/retraction/air supply/water supply control information.

At step S703, the image information is input to the image input section 102. Furthermore, the insertion state information of the insertion section 11 of the endoscope 10 is input to the insertion state input section 103. The condition determination section 104 acquires the image information and insertion state information from the image input section 102 and insertion state input section 103.

At step S704, the condition determination section 104 determines whether or not the improvement of visibility is achieved. The operations of steps S701 through S704 are repeated until it is determined that the improvement of the visibility is achieved. When it is determined that the improvement of the visibility is achieved (“yes” at step S704), the visibility improvement operation returns.

After the visibility improvement operation at step S119, the process returns to step S102.

In the above slack removal, loop disentanglement in the loop handling operation, strain relief, crimp removal, and visibility improvement, if the situation is not improved even after various types of insertion control are conducted, the process may proceed to a halt/alert operation. For instance, the insertion state information and image information may be stored in the storage 107 every time the information is acquired in the operations so that the condition determination section 104 can compare the stored information with the newly acquired insertion state information and image information. Alternatively, it may be determined whether the operations are incomplete even after the insertion control is conducted for a predetermined length of time. In this manner, it may be determined whether or not the process should proceed to the halt/alert operation.

As discussed above, according to the present embodiment, the condition determination section 104 determines whether the condition arises in which the insertion of the insertion section 11 is obstructed. If it is determined that such a condition arises, the drive control section 110 operates the drive sections 20, 30, 40, and 50, based on the insertion control information generated by the insertion control information generation section 105 to improve the condition.

The control method implemented by the control device 100 will be explained below.

For the implementation of the control device 100, a conventional software program (logical control based on algorithmic descriptions) may be adopted, or deep learning or machine learning (e.g., reinforcement learning) may be adopted. With the logical control based on algorithmic descriptions, the operations of the endoscope are controlled by programing. On the other hand, in the reinforcement learning (deep reinforcement learning), image information and insertion state information are used as input data (state), and operations (actions) for individual input items are learned to establish a control model. The deep reinforcement learning is a combination of reinforcement learning and a deep neural network. Such a technique that relates to deep learning and machine learning may be implemented.

For instance, in the case of logical control by programming in the control for loop disentanglement, a combination of the forward/backward operation, rotation operation, and bend operation of the insertion section 11 is described as a program. In contrast, with an artificial intelligence (AI) technique mainly based on the deep learning, a network model is established that employs learning (supervised learning) using experienced surgeons' endoscope manipulations as training data, or reinforcement learning in which a control device conducts self-learning through trial and error. From various types of control information prepared based on such a network model, suitable control information defined in accordance with the image information and insertion state information is selected, thereby achieving automated insertion into the large intestine, which has a complex meandering shape.

Colonoscopy involves manipulations difficult for physicians to acquire so that it may take time for inexperienced physicians to reach the appendix in an examination, or the examination may end up unable to reach the appendix. The examination may also give the patient pain. In contrast, with the insertion operated by the control device 100, an effective and reliable insertion operation can be achieved.

In mechanical automated insertion with a conventional control device, the insertion section often fails to be properly inserted, particularly into the large intestine, which has a complex meandering shape, once some circumstances in which it is difficult to continue the insertion operation arise (e.g., insufficient propulsion conveyance due to slack that appears in the insertion section 11, or load applied to the intestinal tract due to a loop being formed). In contrast, according to the present embodiment, suitable insertion control information is generated by the control device 100 in accordance with the insertion condition, and the insertion operation is controlled by the control device 100 based on this information. In this manner, the present embodiment offers suitable insertion control, such as fully automatic, partially automatic, or semi-automated insertion for an endoscope system incorporating an electric endoscope.

In particular, according to the present embodiment, a smooth insertion operation is performed by identifying various conditions that may make the insertion operation difficult to continue based on various types of information, including image information and insertion state information; generating suitable insertion control information in accordance with the conditions; and conducting control based on this information. Thus, even if the examination target is the large intestine, which has a complex meandering shape, the automatic insertion of the endoscope can be achieved. According to the present embodiment, the basic insertion operations including the forward/backward operation, bend operation, AWS operation, and rotation operation are conducted by the drive sections 20, 30, 40, and 50 and the control device 100 configured to control these drive sections, and therefore various conditions making insertion difficult that may arise during the colonoscopy can be improved in a fully automatic, partially automatic, or semi-automatic manner.

For the condition determination section 104 to determine the conditions, at least one of image information and insertion state information should be used. The conditions can be determined based on one type of the information, without acquiring both of the types of the information. Furthermore, even if both of the types of the information are acquired, both types are not always necessary for the determination of the conditions. By using both types of the information, however, various conditions can be determined. Thus, it is preferable to acquire the two types of the information and use both of them for the determination.

According to the present embodiment, halt/alert control may be implemented, depending on the insertion condition that may make the insertion operation difficult to continue. After the halt or alerting, the surgeon may improve the insertion condition, and the insertion can be suitably achieved through a partially automatic or semi-automated insertion operation.

According to the present embodiment, the inserted shape information is adopted for the detection of slack in the insertion section 11. The slack, however, may be determined, for instance, by arranging sensors on the distal end side and proximal end side of the insertion section 11 and comparing the movement amounts of the distal and proximal end sides obtained by these sensors to find a case of the movement amount on the distal end side that is smaller than the movement amount on the proximal end side (amount of push). Such detection may be combined with detection based on the inserted shape information.

In the above description, the condition determination section 104 is configured to determine the insertion condition based on the insertion state information and image information. In addition to such information, user instruction information input by a user (physician) on an input device that is not shown in the drawings may be used to determine the insertion condition. The user instruction information may be input when the user wishes to verify the insertion condition and immediately perform suitable control upon this condition.

In the above description, a colonoscopy has been used as an example. The endoscope 10, however, is not limited to the large intestine endoscope, and the endoscope system 1 is applicable to various types of endoscopes.

The above conditions classified into (i) to (vii) are introduced merely as examples. In addition to such classification, various situations exist, including changes in the meandering shape of the large intestine due to the insertion section 11 (e.g., partially linearized portion), passage through the bent portion, attachment of the mucous membrane due to suction, and residue, mucus and foams that need to be removed. To deal with such situations, the conditions may be determined and the insertion control information may be generated based on the image information and insertion state information, and additional other types of information. Furthermore, the insertion operation of the insertion section 11 operated by the control device 100 may include operations other than forward/backward, bend, air supply, water supply, suction, and rotation.

According to the present embodiment, an endoscope, a drive device, and a control device are explained as a system. These components, however, may be designed as separate units and used in combination.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
a flexible insertion section to be inserted into an examination target;
an imaging element configured to image the examination target;
a drive section configured to conduct an insertion operation of the insertion section;
an image processing circuit configured to generate image information based on an examination target image obtained by the imaging element;
a condition determination section configured to acquire the image information and insertion state information relating to an insertion state of the insertion section and determine an insertion condition based on the acquired image information and insertion state information;
a storage configured to store the acquired image information and insertion state information;
an insertion control information generation section configured to generate insertion control information based on a determination result obtained by the condition determination section; and
a control section configured to control the drive section based on the insertion control information,
the condition determination section determining whether or not an insertion of the insertion section can be continued by comparing at least one of newly acquired image information and newly acquired insertion state information with corresponding information stored in the storage, and when the condition determination section determines that the insertion cannot be continued, the insertion control information generation section generating halt information.

2. The endoscope system according to claim 1, wherein the condition determination section determines whether or not a condition arises in which the insertion operation of the insertion section is obstructed.

3. The endoscope system according to claim 2, wherein when it is determined that the condition arises in which the insertion operation of the insertion section is obstructed, the control section operates the drive section in a manner such that the obstructing condition can be resolved.

4. The endoscope system according to claim 1, wherein the drive section comprises:
a forward/backward movement mechanism configured to move the insertion section forward and backward, and a forward/backward drive section configured to drive the forward/backward movement mechanism; and
a rotation mechanism configured to rotate the insertion section, and a rotation drive section configured to drive the rotation mechanism,
wherein the insertion control information generation section generates, as the insertion control information, at least one of forward/backward control information and rotation control information.

5. The endoscope system according to claim 4, wherein the condition determination section determines whether or not a loop is formed in the insertion section; when it is determined that a loop is formed, the insertion control information generation section generates the forward/backward control information and the rotation control information in order to resolve the loop; and the control section operates the forward/backward drive section and the rotation drive section based on the forward/backward control information and the rotation control information.

6. The endoscope system according to claim 4, wherein the condition determination section determines whether or not there is slack in the insertion section; when it is determined that there is slack, the insertion control information generation section generates the forward/backward control information and the rotation control information in order to remove the slack; and the control section operates the forward/backward drive section and the rotation drive section, based on the forward/backward control information and the rotation control information.

7. The endoscope system according to claim 4, wherein the condition determination section determines whether or not there is slack in the insertion section; when it is determined that there is slack, the insertion control information generation section generates the forward/backward control information for moving the insertion section backward in order to remove the slack; and the control section operates the forward/backward drive section based on the forward/backward control information.

8. The endoscope system according to claim 4, wherein the condition determination section determines whether or not there is slack in the insertion section; when it is determined that there is slack, the insertion control information generation section generates the forward/backward control information for alternately repeating a forward movement and a backward movement of the insertion section in order to remove the slack; and the control section operates the forward/backward drive section based on the forward/backward control information.

9. The endoscope system according to claim 4, wherein the condition determination section determines whether an external force larger than or equal to a predetermined amount is exerted upon the insertion section; when it is determined that an external force larger than or equal to the predetermined amount is exerted, the insertion control information generation section generates the forward/backward control information and the rotation control information in order to relieve the external force; and the control section operates the forward/backward drive section and the rotation drive section based on the forward/backward control information and the rotation control information.

10. The endoscope system according to claim 4, wherein the drive section comprises:
a bend mechanism configured to bend the insertion section and a bend drive section configured to drive the bend mechanism; and
an air supply mechanism configured to supply air to the insertion section and an air supply drive section to drive the air supply mechanism,
wherein the insertion control information generation section generates, as the insertion control information, at least one of bend control information and air supply control information.

11. The endoscope system according to claim 10, wherein the condition determination section determines whether or not a lumen of the examination target can be identified from the image information; when the lumen of the examination target cannot be identified, the condition determination section determines whether or not the examination target is crimped; when the examination target is crimped, the insertion control information generation section generates at least one of the bend control information and the air supply control information in order to remove a crimp; and the control section operates the bend drive section and the air supply drive section, based on the bend control information and the air supply control information.

12. The endoscope system according to claim 10, wherein the condition determination section determines whether or not a lumen of the examination target can be identified on the image information; when the lumen of the examination target cannot be identified, the condition determination section determines whether or not the examination target is crimped; when the examination target is not crimped, the insertion control information generation section generates at least one of the bend control information, the rotation control information, the forward/backward control information, and the air supply control information in order to improve visibility of an endoscope; and the control section operates the bend drive section, the rotation drive section, the forward/backward drive section, and the air supply drive section based on the bend control information, the rotation control information, the forward/backward control information, and the air supply control information.

13. The endoscope system according to claim 1, wherein the condition determination section determines whether or not the insertion of the insertion section can be continued; when it is determined that the insertion of the insertion section cannot be continued, the insertion control information generation section generates halt information; and the control section halts the drive section based on the insertion control information.

14. The endoscope system according to claim 1, wherein the insertion control information generation section generates the insertion control information in accordance with a control model constituted based on machine learning that adopts, as an input, information relating to at least one of an image and an insertion state.

15. An endoscope control device comprising:
a condition determination section configured to acquire image information and insertion state information relating to an insertion state of an insertion section of an endoscope, and determine an insertion condition based on the acquired information;
a storage configured to store the acquired information;
an insertion control information generation section configured to generate insertion control information for controlling an insertion operation of the insertion section, based on a determination result obtained by the condition determination section; and
a control section configured to control the insertion operation of the insertion section based on the insertion control information,
the condition determination section determining whether or not an insertion of the insertion section can be continued by comparing at least one of newly acquired image information and newly acquired insertion state information with the information stored in the storage; when the condition determination section determines that the insertion cannot be continued, the insertion control information generation section generating halt information.

16. A method of operating an endoscope system that includes a flexible insertion section to be inserted into an examination target, an imaging element configured to image the examination target, and a drive section configured to conduct an insertion operation of the insertion section, the method comprising:
generating image information at an image processing circuit, based on information obtained by the imaging element;
acquiring the image information and insertion state information relating to an insertion state of the insertion section, and determining an insertion condition based on the acquired information, at a condition determination section;
storing the acquired information in a storage;
generating insertion control information at an insertion control information generation section, based on a determination result obtained by the condition determination section;
controlling, at a control section, the drive section based on the insertion control information; and
determining at the condition determination section whether or not an insertion of the insertion section can be continued, by comparing at least one of newly acquired image information and newly acquired insertion state information with the information stored in the storage, and generating halt information at the insertion control information generation section when it is determined at the condition determination section that the insertion cannot be continued.

17. A non-transitory computer-readable storage medium storing a program to cause a computer to function as:
a condition determination section that acquires image information and insertion state information relating to an insertion state of an insertion section of an endoscope, and determines an insertion condition based on the acquired information;
a storage that stores the acquired information;
an insertion control information generation section that generates insertion control information to control an insertion operation of the insertion section based on a determination result obtained by the condition determination section; and
a control section that controls the insertion operation of the insertion section based on the insertion control information,
the program causing the condition determination section to determine whether or not an insertion of the insertion section can be continued by causing the condition determination section to compare at least one of newly acquired image information and newly acquired insertion state information with the information stored in the storage, and when it is determined by the condition determination section that the insertion cannot be continued, the program causing the insertion control information generation section to generate halt information.
